(19) United States
(12) Patent Application Publication
(10) Pub. No.: US 2020/0043567 A1
(43) Pub. Date: Feb. 6, 2020

(54) APPARATUS AND METHODS FOR ASSESSING AN ABILITY OF AN ORGANISM(S) TO METABILIZE TOXID COMPOUNDS

(71) Applicant: Samsung Electronics Co., Ltd., Suwon-si (KR)

(72) Inventors: Tadi Venkata SIVA KUMAR, Bangalore (IN); Rajasekhara Reddy DUVVURU MUNI, Bangalore (IN); Taeyong Kim, Daejeon (KR)

(21) Appl. No.: 16/528,217

(22) Filed: Jul. 31, 2019

(30) Foreign Application Priority Data
Jul. 31, 2018 (IN) 201841028819
Apr. 16, 2019 (KR) 10-2019-0044475

(51) Int. Cl. G16B 5/00; G06N 20/00; G16B 40/00; G16C 20/10; G16C 20/30; G16C 20/70

(52) U.S. Cl. CPC G16B 5/00; G06N 20/00; G16C 20/70; G16C 20/10; G16C 20/30; G16B 40/00

(57) ABSTRACT

An apparatus and methods for assessing an ability of an organism(s) to metabolize toxic compounds, in a computing environment. Embodiments herein relates to the field of metabolic engineering in biochemical pathways and more particularly to an apparatus and methods for assessing an ability of an organism, a strain of the organism or strain of a different organism(s), to metabolize toxic compounds. The method includes receiving an input corresponding to at least one of a metabolite/compound data, at least one reaction data and at least one pathway data. The received input may be assessed to identify toxic compound(s). Further, reactions and pathway(s) may be determined in the identified toxic compound(s). Furthermore, the identified reactions and pathway(s) may be classified as potential toxic generating route(s) or toxic degrading route(s). The assessment can be used for performing comparative assessment of different strains of an organism/different organisms to achieve reaction diversity.

### CROSS-REFERENCE TO RELATED APPLICATION

This application claims the benefits of Indian Patent Application No. 201841028819, filed on Jul. 31, 2018, in the Indian Intellectual Property Office, and Korean Patent Application No. 10-2019-0044475 filed on Apr. 16, 2019, in the Korean Intellectual Property Office, the entire disclosures of which are hereby incorporated by reference.

### BACKGROUND

### 1. Field

The present disclosure relates to the field of metabolic engineering of biochemical pathways and more particularly to an apparatus and methods for assessing an ability of an organism, a strain of the organism, or strain of one or more different organisms, to metabolize toxic compounds.

### 2. Description of the Related Art

Generally, in metabolic engineering, a biochemical processing method such as synthesis or degradation of a metabolite/compound is performed by engineering and optimizing a host organism. The engineering process may involve, for example, removal of native pathway or addition of a non-native pathway into the host organism. Further, understanding organism-level differences in metabolic pathway is crucial to designing a synthetic pathway. For example, some metabolic pathways may have undesired reactions, which may result in toxic metabolites/compounds that are lethal to the organism. Further, some organisms may possess the metabolic pathways that can effectively metabolize toxins to a non-toxic metabolite(s)/compound(s). Such metabolic pathways may also vary across different strains of a particular organism.

Conventional methods of assessing the toxicity of a metabolite/compound with respect to an organism involve measuring the growth of organism in presence of that particular metabolite/compound. Conventional methods are not well-suited to identify the particular mechanism of toxicity of the metabolite/compound. Furthermore, the conventional methods may not predict the ability to metabolize the toxins and may also be unable to suggest an alternate pathway.

### SUMMARY

One aspect of the invention provides an apparatus and method for assessing, in a computing environment, the ability of an organism to metabolize toxic compounds.

Another aspect of the invention provides an apparatus and method to predict the nature of a biochemical reaction with respect to its ability to degrade or synthesize at least one toxic compound.

Another aspect of the invention provides methods of identifying reaction level differences in a pathway between different organisms or different strains of an organism corresponding to a metabolite/compound.

Another aspect of the invention provides an apparatus and method to analyze metabolic pathways based on toxicity features, and the use of such an apparatus or method for identifying at least one of a lethal pathway and a degradation pathway.

Another aspect of the invention provides an apparatus and method for suggesting an alternative metabolic pathway that may be non-toxic.

Certain embodiments provide a processor-implemented method for assessing an ability of one or more organism(s) to metabolize at least one toxic compound, in a computing environment. The method includes receiving, by an electronic device, input data corresponding to at least one biochemical compound data, wherein the at least one biochemical compound data comprises compound data, at least one reaction data, and at least one pathway data. The method includes extracting, by the electronic device, the compound data associated with the received at least one reaction data and the at least one pathway data. The method includes retrieving, by the electronic device, a molecular information corresponding to the extracted compound data, from a database associated with the electronic device. The retrieved molecular information is used by the electronic device to generate a plurality of first features comprising identifying at least one of constitutional data, topological data, electronic data, and fingerprint data. The electronic device then identifies toxic data by mapping the plurality of generated first features with at least one second features stored in the database associated with the electronic device. The method includes assessing, by the electronic device, an effect of toxicity of the compound data on the at least one reaction data and the at least one pathway data, based on the identified toxic data, wherein assessing the effect of toxicity comprise determining the lethality of the compound data to at least one of, an organism, a strain of the organism, and a strain of a different organism.

Certain embodiments herein provide an apparatus for assessing ability of an organism(s) to metabolize at least one toxic compound, in a processor-mediated environment. The apparatus includes at least one processor and at least one memory unit coupled to the processor. The apparatus is configured to receive an input corresponding to at least one biochemical compound data, wherein the at least one biochemical compound data comprises at least one of, a compound data, at least one reaction data, and at least one pathway data. The apparatus is configured to extract the compound data associated with the received at least one reaction data and the at least one pathway data. The apparatus is configured to retrieve a molecular information corresponding to the extracted compound data, from a database associated with the electronic device. The apparatus is configured to generate a plurality of a first features corresponding to the retrieved molecular information, wherein the first features comprises at least one of a constitutional data, a topological data, an electronic data, and a fingerprint data. The apparatus is configured to identify a toxic data of the at least one biochemical compound data based on mapping the plurality of the generated first features with at least one second features stored in the database associated with the electronic device. The apparatus is configured to assess an effect of toxicity of the compound data, on the at least one reaction data and the at least one pathway data, based on the identified toxic data, wherein assessing the effect of toxicity comprise determining the lethality of the compound data to at least one of, an organism, a strain of the organism and a strain of a different organism.

Additional aspects will be set forth in part in the description which follows and, in part, will be apparent from the description, or may be learned by practice of the presented exemplary embodiments.

### BRIEF DESCRIPTION OF THE DRAWINGS

Embodiments herein are illustrated in the accompanying drawings, throughout which like reference letters indicate corresponding parts in the various figures. The embodiments herein will be better understood from the following description with reference to the drawings, in which:

FIG. 1 illustrates a block diagram of an apparatus for assessing an ability of an organism and a strain of the organism or a strain of a different organisms, to metabolize toxic compounds, in a computing environment, according to embodiments as disclosed herein;

FIG. 2 illustrates a detailed view of a processing module as shown in FIG. 1, comprising various modules, according to embodiments as disclosed herein;

FIG. 3A illustrates a schematic diagram of example scenario for identification of pathways that can degrade toxins in a given organism and a strain of the organism or a strain of a different organisms, according to embodiments as disclosed herein;

FIG. 3B illustrates a schematic diagram of a pathway assessment using an example scenario, according to embodiments as disclosed herein;

FIG. 3C illustrates a schematic diagram of classification of the pathway, according to embodiments as disclosed herein;

FIG. 4 is a schematic diagram of method for identifying the difference in a strain specific toxic pathway, according to embodiments as disclosed herein;

FIG. 5A is a flow chart of a method for assessing ability of an organism and a strain of the organism or a strain of a different organisms to metabolize at least one toxic compound, in a computing environment, according to the embodiments as disclosed herein;

FIG. 5B is a flow chart of a method for outputting an ability data and suggestion data of at least one biochemical compound data to degrade and synthesize at least one toxic compounds, according to embodiments as disclosed herein;

FIG. 5C is a flow chart of a method for creating and storing learned data as a function, according to embodiments as disclosed herein;

FIG. 5D is a flow chart of a method for determining the toxicity using the stored function, according to embodiment as disclosed herein; and

FIG. 6 illustrates a computing environment implementing an apparatus and methods for assessing an ability of an organism and a strain of the organism or a strain of a different organism(s), to metabolize toxic compounds, according to embodiments as disclosed herein.

### DETAILED DESCRIPTION

The example embodiments herein and the various features and advantageous details thereof are explained more fully with reference to the non-limiting embodiments that are illustrated in the accompanying drawings and detailed in the following description. Descriptions of well-known components and processing techniques are omitted so as to not unnecessarily obscure the embodiments herein. The description herein is intended merely to facilitate an understanding of ways in which the example embodiments herein can be practiced and to further enable those of skill in the art to practice the example embodiments herein. Accordingly, this disclosure should not be construed as limiting the scope of the example embodiments herein.

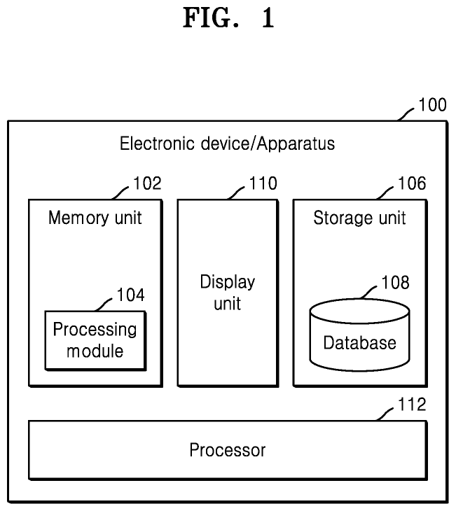

The embodiments herein achieve an apparatus and processor-implemented methods for assessing an ability of one or more organism(s) to metabolize toxic compounds, in a computing environment. Referring now to the drawings, and more particularly to FIGS. 1 through 6, where similar reference characters denote corresponding features consistently throughout the figures, there are shown example embodiments.

FIG. 1 illustrates a block diagram of an apparatus 100 for assessing an ability of an organism and a strain of the organism or a strain of a different organism(s) to metabolize toxic compounds, in a processor-mediated environment, according to embodiments as disclosed herein.

The apparatus 100 includes a memory unit 102, a storage unit 106, a display unit 110, and a processor 112. Further, the apparatus 100 may include a processing module 104. When the machine readable instructions are executed, the processing module 104 causes the apparatus 100 to process the data in the computing environment. Furthermore, the apparatus 100 includes a database 108 to store the required data. The apparatus 100 may occasionally connect to the server (not shown) via a communication network (not shown). The communication network may be a wired (such as a local area network, Ethernet, and so on) or a wireless communication network (such as Wi-Fi, Bluetooth, and so on). The apparatus 100 may also retrieve the data from the external databases (not shown) as needed and store the retrieved data in the local database 108 associated with the apparatus 100. The apparatus 100 can extract data from the database (not shown) and can launch simulations, for example, in response to a query or command received by the apparatus 100 or remote server (not shown). Examples of databases that can be accessed by the apparatus 100 includes at least one of a compound database, a gene database, a reaction database, a bio-particle database, a reference database, and so on. The database 108 associated with the apparatus 100 can be represented in a markup language format, which allows databases and application tools to exchange information. The markup language format, for example includes, Standard Generalized Markup Language (SGML), Hypertext markup language (HTML), Extensible Markup language (XML), Systems Biology Markup Language (SBML), Systems Biology Ontology (SBO), Biological Pathway Exchange Language (BioPAX), and so on. Although not shown, the apparatus 100 can be connected to a cloud computing platform via a gateway.

Further, the apparatus 100 can also be referred herein as an electronic device 100. The apparatus 100/electronic device 100 can be, but not limited to, a mobile phone, a smart phone, a tablet, a handheld device, a phablet, a laptop, a computer, a wearable computing device, and so on. The apparatus 100 may comprise other components such as input/output interface, communication interface and so on. The apparatus 100 may comprise a user application interface (not shown), an application management framework (not shown), and an application framework (not shown) for assessing an ability of an organism and a strain of the organism or a strain of a different organism(s) to metabolize toxic compounds. The application framework may comprise different modules and sub modules to execute the operation for assessing an ability of an organism and a strain of the organism or a strain of a different organism(s) to metabolize toxic compounds. The application framework can be, for example, a software library that provides a fundamental structure to support the development of applications for a specific environment. The application framework may also be used in developing graphical user interface (GUI) and web-based applications. Further, the application management framework may be responsible for the management and maintenance of the application and definition of the data structures used in databases and data files.

In one embodiment, the methods herein may be implemented using the apparatus 100. Thus, one or more (or all) steps of the method can be carried out by or with the assistance of a computer processor. The embodiments herein may perform specified manipulations of data or information in response to a command or set of commands provided by a user. In alternative embodiment, the methods herein may be implemented using apparatus 100 such as server (not shown). The server may be implemented using apparatus 100.

In another embodiment, the methods herein may be implemented partly using client device (not shown) and partly using server. The client device can be the electronic device 100 or apparatus 100 and the server can be a remote server or cloud server, wherein the client device and the server is communicatively coupled to establish a communication session. The methods herein can be performed in a sequential manner by the combination of client device and server.

Accordingly, the apparatus 100 may allow the user to input at least one biochemical compound data as desired by the user. Biochemical compound data is data that pertains to a given biochemical compound. In an example, the apparatus 100 may select an approach such as chemical or biochemical approach for synthesis/degradation based on selecting the enzyme related to a metabolic reaction of the biochemical compound. Further, a well-known knowledge database maybe used to predict chemical reactions using reaction rules. The ability of an organism and a strain of the organism or a strain of a different organism(s) to metabolize toxins may be identified based on toxicity-based refinement of the data. The pathways for biochemical synthesis may be accessed and provide a score based on structural moiety (i.e. part of molecule) based refinement. Also, the pathways for bio-chemicals synthesis may be accessed and provide a score based on transformation association scoring. The host organism may be selected for biochemical processing and enzyme flexibility assessment may be performed. Also, carbon retention and yield estimation may be performed to predict the toxicity.

In another embodiment, the apparatus 100 may be configured to receive an input corresponding to the at least one biochemical compound data. In an embodiment, the at least one biochemical compound data includes at least one of a metabolite/compound data, at least one reaction data and at least one pathway data. In an embodiment, the apparatus 100 may be configured to extract the compound data associated with the received at least one reaction data and the at least one pathway data. In an embodiment, the apparatus 100 may be configured to retrieve molecular information corresponding to the extracted compound data from a database 108 associated with the electronic device 100. In an embodiment, the apparatus 100 may be configured to generate plurality of a first features corresponding to the retrieved molecular information. In an embodiment, the apparatus 100 may be configured to identify a toxic data of at least one biochemical compound data based on mapping plurality of the generated first features with at least one second features stored in the database 108 associated with the electronic device 100.

In another embodiment, the first features and second features may include at least one of, but not limited to, a constitutional data, a topological data, an electronic data, and a fingerprint data as they relate to one or more biochemical compounds. The constitutional data may include at least one of, but not limited to, an A Log P (i.e. Atom based Partition coefficient), an acid group count, an aromatic atom count, an aromatic bond count, a basic group count, a bond count, an element count, and a largest chain and so on. The topological data may include at least one of, but not limited to, carbon types, chi chain indices, eccentric connectivity index, hybridization ratio, small ring descriptor, topological polar surface area, and so on. The electronic data may include at least one of, but not limited to, anatomic polarizabilities, bond polarizabilities, charged partial surface areas, hydrogen bond acceptors, hydrogen bond donors, and so on. The fingerprint data may include at least one of, but not limited to, a circular fingerprint, an extended fingerprint, an extended connectivity fingerprints (ECFPs), a Molecular ACCess System (MACCS) fingerprint, and so on.

In an embodiment, the apparatus 100 may be configured to assess the toxicity of the compound data on the at least one reaction data and the at least one pathway data based on the identified toxic data. In an embodiment, assessing the effect of toxicity includes determining the lethality of the compound data to at least one of an organism and a strain of the organism.

In an embodiment, the apparatus 100 may be configured to determine at least one of a toxin degradation data, a toxin synthesis data and a toxin route data, corresponding to the at least one reaction data and the at least one pathway data, based on identifying the toxic data of the at least one biochemical compound data. In an embodiment, the apparatus 100 may be configured to analyze the determined toxin degradation data, toxin synthesis data and/or toxin route data, corresponding to the at least one biochemical compound data. In an embodiment, the apparatus 100 may be configured to output an ability data of the at least one biochemical compound data to degrade the at least one toxic compound, based on the reaction data of at least one of the toxin degradation data, the toxin synthesis data and the toxin route data. In an embodiment, the apparatus 100 may be configured to output an ability data of the at least one biochemical compound data to synthesize the at least one toxic compounds, based on the analyzed reaction data of the at least one of the toxin degradation data, the toxin synthesis data and the toxin route data. In an embodiment, outputting the ability data comprise determining the capability of the at least one biochemical compound data to metabolize the at least one toxic compounds. In an embodiment, outputting the ability data comprise determining an existence of the route between the compounds. In an embodiment, the apparatus 100 may be configured to output a suggestion data corresponding to an alternative reaction route within the provided at least one biochemical compound data. In an embodiment, the suggestion data is outputted based on the analyzed at least one of the toxin degradation data, the toxin synthesis data and the toxin route data corresponding to the at least one biochemical compound data. In an embodiment, outputting the suggestion data includes identifying the at least one of a non-toxic route between the compounds.

In an embodiment, the apparatus 100 may be configured to select at least one appropriate feature related to toxicity, in the generated plurality of the first features corresponding to the at least one biochemical compound data. In an embodiment, selecting the appropriate features related to toxicity includes reducing the first features using dimensionality reduction method.

In an embodiment, the apparatus 100 may be configured to create a function associated with a learned data corresponding to the selected appropriate features for determining the toxicity of the at least one biochemical compound data. In an embodiment, creating the function comprises computing a mathematical function derived by linear combination of the stored second features retrieved from the database 108. In an embodiment, the apparatus 100 may be configured to store in the database 108, the created function, to determine the toxicity of subsequent the at least one biochemical compound data. The function can be a Radial Basis Function using a Support Vector Machine (SVM) method. The function can be, at least one of, but not limited to,

$$K ( x , {x}^{′} ) = exp ( - \frac{{‖ x - {x}^{′} ‖}^{2}}{2 {σ}^{2}} )$$

Where, the ‘∥x−x′∥²’ may be recognized as the squared Euclidean distance between the two feature vectors, the ‘σ’ is a free parameter, the ‘exp’ is an exponential function.

In an embodiment, the apparatus 100 may be configured to receive the input corresponding to the at least one biochemical compound data. In an embodiment, the apparatus 100 may be configured to insert combination of the appropriate features corresponding to the at least one biochemical compound data, in the stored function. In an embodiment, the apparatus 100 may be configured to determine the toxicity of the at least one biochemical compound data, based on inserting combination of the appropriate features in the stored function. In an embodiment, the apparatus 100 may be further configured to analyze a reaction data of the determined at least one of the toxin degradation data, the toxin synthesis data and the toxin route data, corresponding to the at least one biochemical compound data. In an embodiment, the reaction data comprises a reaction level difference in pathways between a strains associated with organisms or different organisms corresponding to the metabolite/compound.

In an embodiment, extracting the at least one compound data associated with the received input corresponding to the at least one biochemical compound data includes breaking down the at least one biochemical compound data into compounds. In an embodiment, generating the plurality of the first features includes identifying at least one of the constitutional data, the topological data, the electronic data, and the fingerprint data. The constitutional data may include at least one of, but not limited to, data related to number of carbon atoms, single bond data, double bond data, and so on. The topological data may include at least one of, but not limited to, data related to length of chain, volume, and so on. The electronic data may include at least one of, but not limited to, data relative positive charge and negative charge of the atoms, and so on. The fingerprint data may include at least one of, but not limited to, data related to parts of molecule, bit fingerprint, count fingerprint, and so on.

In an embodiment, determining the at least one of the toxin degradation data, the toxin synthesis data and the toxin route data comprises, identifying a reaction level differences in pathways between a strain associated with organisms. In an embodiment, the strain comprises variation of a species associated with the organism. In an embodiment, outputting the ability data of the at least one biochemical compound data to degrade or synthesize at least one toxic compounds comprises determining the capability of the at least one biochemical compound data to metabolize the at least one toxic compounds and existence of the route between the metabolites/compounds. In an embodiment, suggesting the alternative reaction route within the provided at least one biochemical compound data, based on the analyzed reaction data of the at least one toxic compounds comprises identifying the at least one of a non-toxic route, between the metabolites/compounds. In an embodiment, the mathematical function may be derived using at least one of a Random Forest Method (RFM) and the Support Vector Machine (SVM) method.

The diagram of FIG. 1 illustrates functional components of the computer implemented system. In some cases, the component may be a hardware component, a software component, or a combination of hardware and software. Some of the components may be application level software, while other components may be operating system level components. In some cases, the connection of one component to another may be a close connection where two or more components are operating on a single hardware platform. In other cases, the connections may be made over network connections spanning long distances. Each embodiment may use different hardware, software, and interconnection architectures to achieve the functions described.

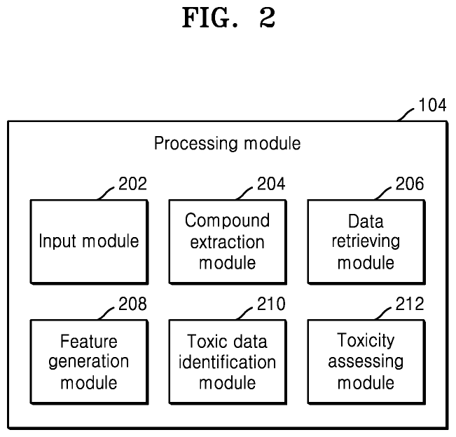

FIG. 2 illustrates a detailed view of processing module 104 as shown in FIG. 1, comprising various modules, according to embodiments as disclosed herein. In an embodiment, the apparatus 100 may comprise a processing module 104 stored in the memory unit 102 (as shown in FIG. 1). The processing module 104 may comprise plurality of sub modules. The plurality of sub modules can comprise of, an input module 202, a compound extraction module 204, a data retrieving module 206, a feature generation module 208, a toxic data identification module 210, and a toxicity assessing module 212.

In an embodiment, the input module 202 may receive an input corresponding to at least one biochemical compound data. In an embodiment, the at least one biochemical compound data includes at least one of a metabolite/compound data, at least one reaction data, at least one pathway data, and so on. In an embodiment, the compound extraction module 204 may extract the compound data associated with the received at least one reaction data and the at least one pathway data. In an embodiment, the data retrieving module 206 may retrieve a molecular information corresponding to the extracted the compound data, from a database 108 associated with the electronic device 100. In an embodiment, the feature generation module 208 may generate plurality of the first features corresponding to the retrieved molecular information. In an embodiment, generating the plurality of the first features includes identifying the at least one of a constitutional data, a topological data, an electronic data, and a fingerprint data. In an embodiment, the toxic data identification module 210 may identify a toxic data of the at least one biochemical compound data based on mapping plurality of the generated first features with at least one second feature stored in the database 108 associated with the electronic device 100. In an embodiment, the toxicity assessing module 212 may assess an effect of toxicity of the compound data, on the at least one reaction data and the at least one pathway data, based on the identified toxic data. In an embodiment, assessing the effect of toxicity includes determining the lethality of the compound data to at least one of, an organism and a strain of the organism.

The embodiments herein can comprise hardware and software elements. The embodiments that are implemented in software include but are not limited to, firmware, resident software, microcode, etc. The functions performed by various modules described herein may be implemented in other modules or combinations of other modules. For the purposes of this description, a computer-usable or computer readable medium can be any apparatus that can comprise, store, communicate, propagate, or transport the program for use by or in connection with the instruction execution system, apparatus, or device.

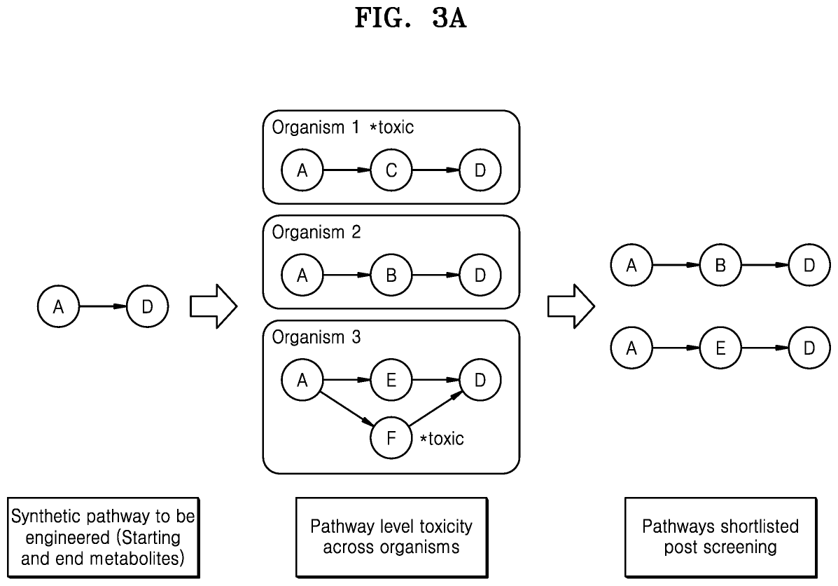

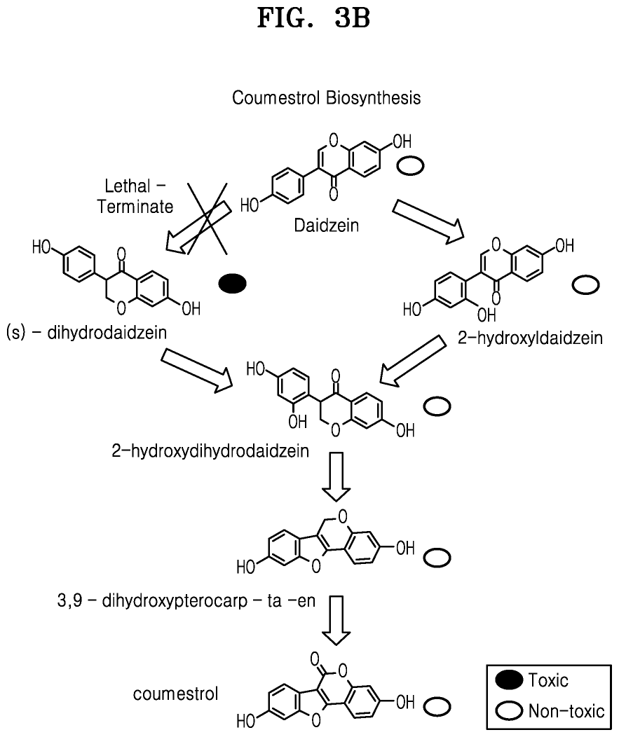

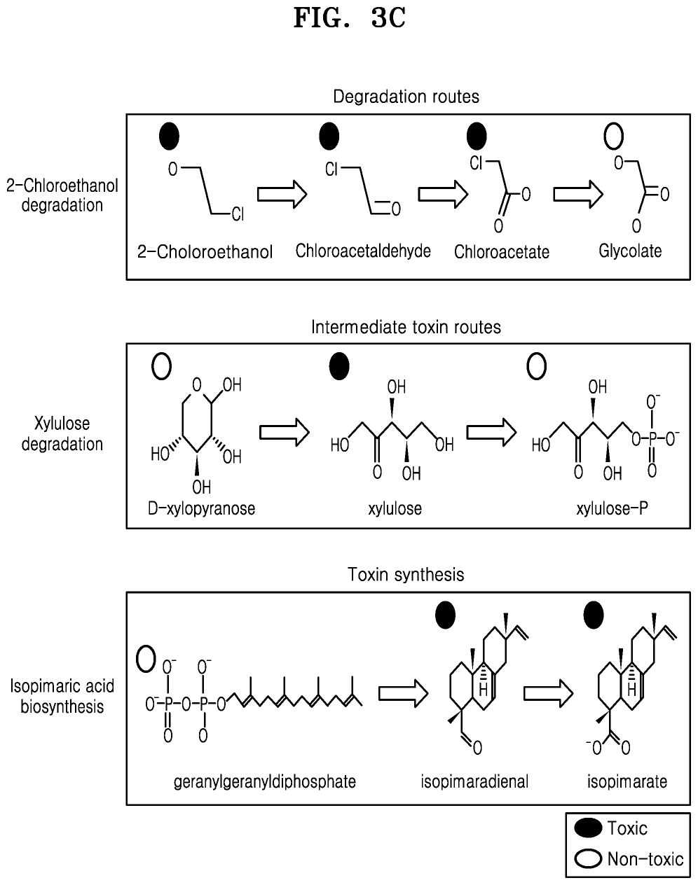

FIG. 3A illustrates a schematic diagram of an example scenario for identification of pathways that can degrade toxins in a given organism and a strain of the organism or a strain of the different organisms, according to embodiment as disclosed herein.

In an example, if the user inputs the at least one biochemical compound data such as A, B, C, D, E and F, wherein A is an initial metabolite/compound and D is an end product. The processing module 104 may process the data according to the inputted at least one biochemical compound data. The processing module 104 may determine, if the routes from A to D with intermediates B, C, E and F, across the organism and a strain of the organism or a strain of a different organisms, are toxic or non-toxic, based on retrieving the pathway data from the database 108. The processing module 104 may output the preferred pathway and alternative pathway which are non-toxic.

FIG. 3B illustrates a schematic diagram of example scenario for pathway assessment, according to embodiments as disclosed herein.

In an example, the pathway assessment may be performed for the biosynthesis pathway such as Coumestrol biosynthesis. The database 108 may be accessed to retrieve the data regarding the existing biochemical pathways and toxicity data. As shown in the FIG. 3, the Daidzein molecule may take both toxic and non-toxic pathway based on the provided at least one biochemical compound data. The apparatus 100 may predict and suggest the non-toxic pathway. For example, Daidzein molecule metabolized with (s)—Dihydrodaidzein molecule is a toxic route, and Daidzein metabolized with 2-Hydroxyl daidzein is a non-toxic route. If the molecule is not able to metabolize with either of the molecule, then the apparatus 100 may output the non-ability of the molecule to metabolize with the at least one toxic compounds. The biochemical pathways may be classified based on the metabolite/compound toxicity. The classification can be at least one of toxin degradation, toxin synthesis and intermediate toxin routes.

FIG. 3C illustrates a schematic diagram of classification of the pathway, according to embodiments as disclosed herein. In an example, if the metabolizing of one or more metabolite/compound or reactants is toxic and the product is non-toxic, then the pathway is a degradation pathway. Further, if the base reactant or metabolite/compound is non-toxic and metabolizing of one or more compounds or products is a toxic, then the route is a toxin synthesis pathway. Furthermore, if the reactants and products are non-toxic, wherein the intermediate metabolizing of one or more metabolite/compound or reactants is toxic, then the route is an intermediated toxin pathway.

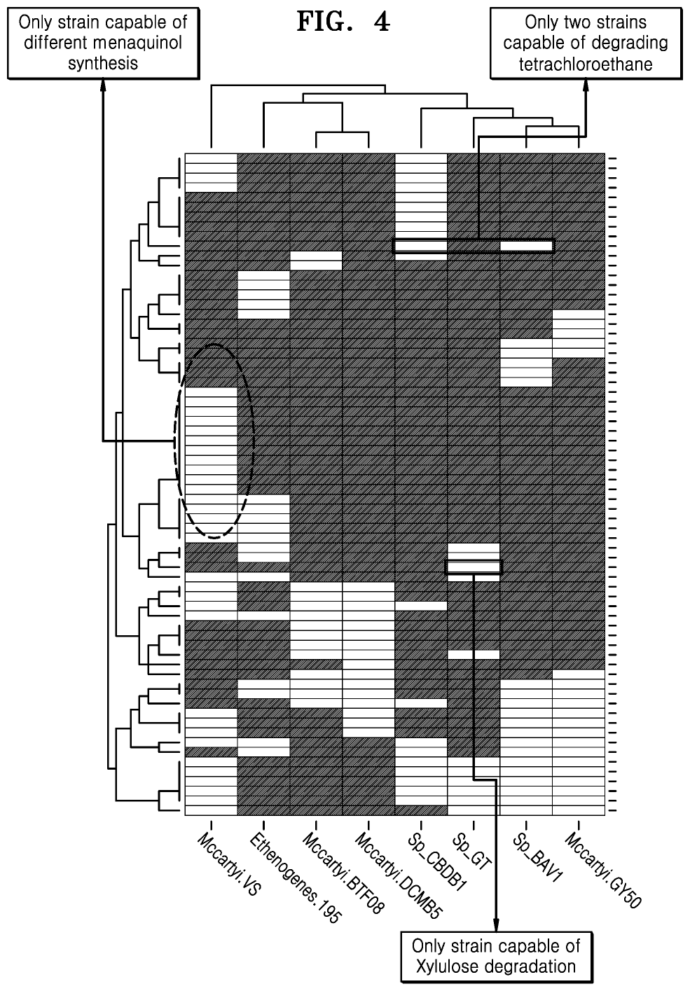

FIG. 4 is a schematic diagram of method for identifying the difference in a strain specific toxic pathway, according to embodiments as disclosed herein. In an example, consider a species such as Dehalococcoidesmccartyi that has characteristics of strict organohalide respiring bacteria with reductive dehalogenation capacity. The Dehalococcoidesmccartyi may have an eight different strains or variation in organisms/strains of the different organisms. The capability related to degradation pathway may be found in the Dehalococcoidesmccartyi species such as two strains are capable of degrading tetrachloro ethane and one strain capable of Xylulose degradation. Further, capability related to toxin synthesis may be found in the Dehalococcoidesmccartyi species such as only one strain is capable of different Menaquinol synthesis. The toxicity determination module 212 associated with the apparatus 100 may output specific strains of the same species capable of degrading specific toxin compounds.

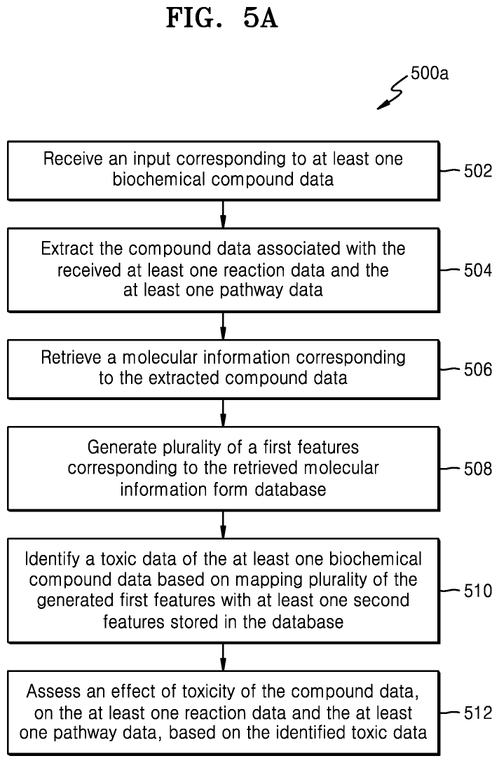

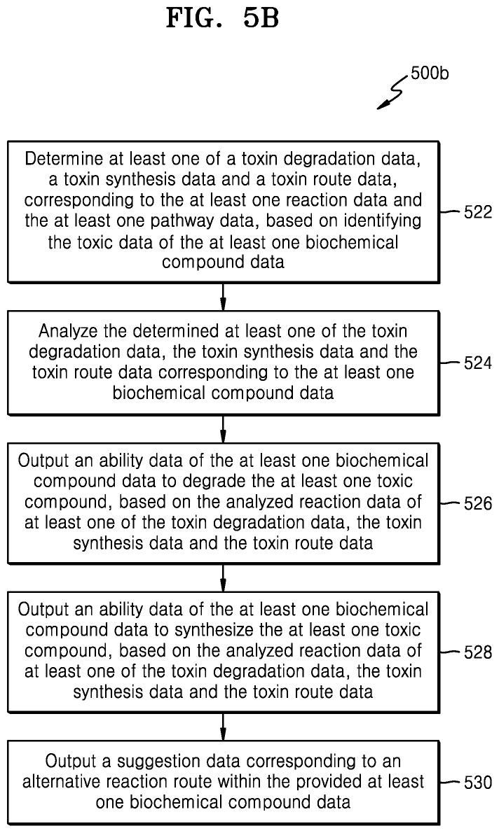

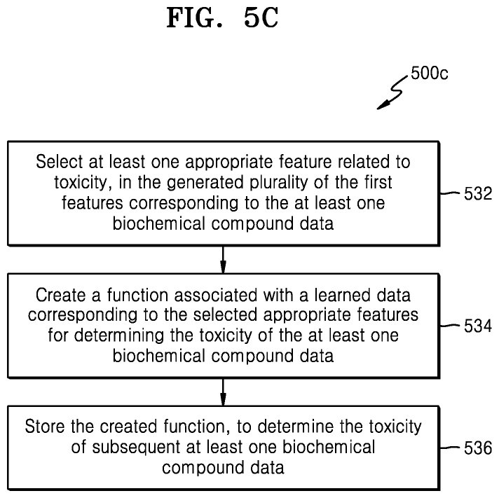

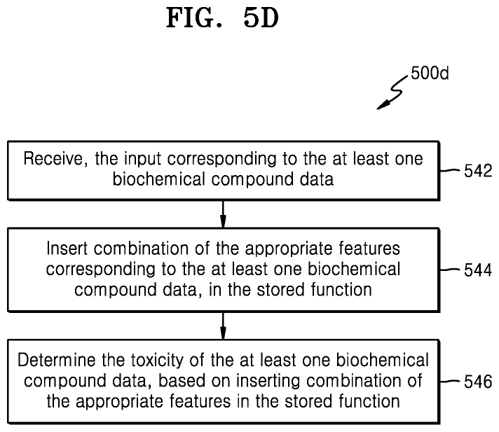

FIG. 5A is a flow chart of the method 500a for assessing ability of an organism and a strain of the organism or a strain of the different organisms, to metabolize at least one toxic compound, in a computing environment, according to the embodiments as disclosed herein.

At step 502, the method 500a includes receiving, by an electronic device 100, an input corresponding to at least one biochemical compound data. In an embodiment the at least one biochemical compound data includes at least one of, a metabolite/compound data, at least one reaction data and at least one pathway data. At step 504, the method 500a includes extracting, by the electronic device 100, the compound data associated with the received at least one reaction data and the at least one pathway data. At step 506, the method 500a includes, retrieving, by the electronic device 100, a molecular information corresponding to the extracted compound data, from a database 108 associated with the electronic device 100. At step 508, the method 500a includes generating, by the electronic device 100, plurality of a first features corresponding to the retrieved molecular information. In an embodiment, generating the plurality of the first features includes identifying the at least one of a constitutional data, a topological data, an electronic data, and a fingerprint data. At step 510, the method 500a includes identifying, by the electronic device 100, a toxic data of at least one biochemical compound data based on mapping plurality of the generated first features with at least one second features stored in the database 108 associated with the electronic device 100. At step 512, the method 500a includes assessing, by the electronic device (100), an effect of toxicity of the compound data, on the at least one reaction data and at least one pathway data, based on the identified toxic data. In an embodiment, assessing the effect of toxicity comprises determining the lethality of the compound data to at least one of, an organism and a strain of the organism.

The various actions in method 500a may be performed in the order presented, in a different order or simultaneously. Further, in some embodiments, some actions listed in FIG. 5A may be omitted.

FIG. 5B is a flow chart of the method 500b for outputting an ability data and suggestion data of the at least one biochemical compound data to degrade and synthesize at least one toxic compounds, according to embodiments as disclosed herein.

At step 522, the method 500b includes, determining, by the electronic device (100), at least one of a toxin degradation data, a toxin synthesis data and a toxin route data, corresponding to the at least one reaction data and the at least one pathway data, based on identifying the toxic data of at least one biochemical compound data. At step 524, the method 500b includes, analyzing, by the electronic device (100), the determined toxin route data corresponding to the at least one biochemical compound data. At step 526, the method 500b includes outputting, by the electronic device 100, an ability data of the at least one biochemical compound data to degrade the at least one toxic compound, based on the analyzed reaction data of at least one of the toxin degradation data, the toxin synthesis data and the toxin route data. At step 528, the method 500b includes outputting by the electronic device 100, the ability data of the at least one biochemical compound data to synthesize at least one toxic compound, based on the analyzed reaction data of the at least one of the toxin degradation data, the toxin synthesis data and the toxin route data. In an embodiment, outputting the ability data includes determining the capability of the at least one biochemical compound data to metabolize the at least one toxic compounds. In an embodiment, outputting the ability data include determining an existence of the route between the compounds. At step 530, the method 500b includes outputting, by the electronic device 100, a suggestion data corresponding to an alternative reaction route within the provided at least one biochemical compound data. In an embodiment, wherein the suggestion data is outputted based on the analyzed reaction data of the at least one toxic compounds. In an embodiment, outputting the suggestion data includes identifying the at least one of a non-toxic route between the compounds.

The various actions in method 500b may be performed in the order presented, in a different order or simultaneously. Further, in some embodiments, some actions listed in FIG. 5B may be omitted.

FIG. 5C is a flow chart of the method 500c for creating and storing learned data as a function, according to embodiments as disclosed herein.

At step 532, the method 500c includes, selecting, by the electronic device 100, at least one appropriate feature related to toxicity, in the generated plurality of the first features corresponding to the at least one biochemical compound data. In an embodiment, selecting the appropriate features related to toxicity includes reducing the first features using dimensionality reduction method. At step 534, the method 500c includes, creating by the electronic device 100, a function associated with a learned data corresponding to the selected appropriate features for determining the toxicity of the at least one biochemical compound data. In an embodiment creating the function includes computing a mathematical function derived by linear combination of the stored second features retrieved from the database 108. At step 536, the method 500c includes, storing by the electronic device 100, in the database 108, the created function, to determine the toxicity of subsequent at least one biochemical compound data.

The various actions in method 500c may be performed in the order presented, in a different order or simultaneously. Further, in some embodiments, some actions listed in FIG. 5C may be omitted.

FIG. 5D is a flow chart of the method 500d for determining the toxicity using the stored function, according to embodiment as disclosed herein.

At step 542, the method 500d includes, receiving, by the electronic device 100, the input corresponding to the at least one biochemical compound data. At step 544, the method 500d includes inserting, by the electronic device 100, combination of the appropriate features corresponding to the at least one biochemical compound data, in the stored function. At step 546, the method 500d includes determining, by the electronic device 100, the toxicity of the at least one biochemical compound data.

The various actions in method 500d may be performed in the order presented, in a different order or simultaneously. Further, in some embodiments, some actions listed in FIG. 5D may be omitted.

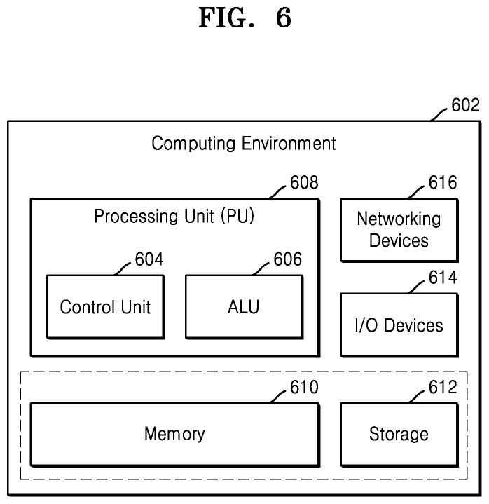

FIG. 6 illustrates a computing environment 602 implementing an apparatus and methods for assessing an ability of the organism and a strain of the organism or the strain of the different organisms, to metabolize toxic compounds, according to embodiments as disclosed herein.

As depicted in the figure, the computing environment 602 comprises at least one processing unit 608 that is equipped with a control unit 604 and an Arithmetic Logic Unit (ALU) 606, a memory 610, a storage unit 612, plurality of networking devices 616 and plurality Input output (I/O) devices 614. The processing unit 608 is responsible for processing the instructions of the scheme. The processing unit 608 receives commands from the control unit in order to perform its processing. Further, any logical and arithmetic operations involved in the execution of the instructions are computed with the help of the ALU 606. The overall computing environment 602 can be composed of multiple homogeneous or heterogeneous cores, multiple CPUs of different kinds, special media and other accelerators. The processing unit 608 is responsible for processing the instructions of the scheme. Further, the plurality of processing units 608 may be located on a single chip or over multiple chips.

The scheme comprising of instructions and codes required for the implementation are stored in either the memory unit 610 or the storage 612 or both. At the time of execution, the instructions may be fetched from the corresponding memory 610 or storage 612, and executed by the processing unit 608.

In case of any hardware implementations various networking devices 616 or external I/O devices 614 may be connected to the computing environment to support the implementation through the networking unit and the I/O device unit.

In an embodiment, the computing environment may be at least one of an electronic device, server, client device, and so on. The computing environment 602 may perform assessing an ability of an organism and the strain of the organism or a strain of the different organisms, to metabolize toxic compounds. The computing environment may include the application management framework. The application management framework may include plurality of processing modules 104 and sub modules. The processing modules 104 may be stored in the memory 610 of the storage unit 612. The processing modules 104 may be responsible for execution of the task for assessing an ability of an organism and the strain of the organism or a strain of the different organisms, to metabolize toxic compounds.

Further, the processing module 104 may be configured to identify and aggregate the metabolites/compounds that are experimentally validated to be toxic/non-toxic to microbes which may result in accurately predicting the toxicity of any given metabolite/compound. The processing module 104 may encode and identify molecular descriptors which could be transformed into a function for accurate prediction of metabolite/compound toxicity. The processing module 104 may also be configured to identify organism/strain specific pathways and alternate reaction routes in the pathways along with selection of non-toxic routes. The processing module 104 may be configured to identify reaction level differences in the pathways between strains and identify toxic routes that may or may not be handled by an organism/strain.

The embodiments disclosed herein can be implemented through at least one software program running on at least one hardware device and performing network management functions to control the elements. The elements shown in FIG. 1 can be at least one of a hardware device, or a combination of hardware device and software module.

What is claimed is:

1. A processor-implemented method for assessing ability of an organism to metabolize at least one toxic compound, the method comprising:
receiving, by an electronic device, an input of biochemical compound data, wherein the at least one biochemical compound data comprises compound data, at least one reaction data, and at least one pathway data;
extracting, by the electronic device, compound data associated with the received at least one reaction data and the at least one pathway data;
retrieving, by the electronic device, molecular information corresponding to the extracted compound data from a knowledge database of the electronic device;
generating, by the electronic device, a plurality of first features corresponding to the retrieved molecular information, wherein the first features comprise at least one of constitutional data, topological data, electronic data, and fingerprint data;
identifying, by the electronic device, toxic data of the at least one biochemical compound data, based on mapping the plurality of the generated first features with at least one of a set of second features stored in the database associated with the electronic device; and
assessing, by the electronic device, toxicity of the compound data on the at least one reaction data and the at least one pathway data, based on the identified toxic data,
wherein assessing the toxicity comprises determining the lethality of the compound data to an organism.

2. The method of claim 1, wherein the method further comprises:
determining, by the electronic device, at least one of toxin degradation data, toxin synthesis data, and toxin route data, corresponding to the at least one reaction data and the at least one pathway data based on the identified toxic data;
analyzing, by the electronic device, the toxin degradation data, toxin synthesis data, and/or toxin route data;
outputting, by the electronic device, ability data indicating the ability of the at least one biochemical compound data to degrade the at least one toxic compound, based on the analyzed toxin degradation data, toxin synthesis data, and/or toxin route data;
outputting, by the electronic device, ability data indicating the ability of the at least one biochemical compound data to synthesize the at least one toxic compound, based on the analyzed toxin degradation data, toxin synthesis data, and/or toxin route data, wherein outputting the ability data comprises determining the capability of the at least one biochemical compound data to metabolize the at least one toxic compound, wherein outputting the ability data comprise determining an existence of the route between the compounds; and
outputting, by the electronic device, suggestion data corresponding to an alternative reaction route within the provided at least one biochemical compound data, wherein the suggestion data is outputted based on the analyzed toxin degradation data, toxin synthesis data, and/or toxin route data corresponding to the at least one biochemical compound data, wherein outputting the suggestion data comprise identifying at least one non-toxic route between the biochemical compounds.

3. The method of claim 1, wherein the method further comprises:
selecting, by the electronic device, at least one toxicity feature from the generated plurality of the first features corresponding to the at least one biochemical compound data, wherein selecting at least one toxicity feature comprises reducing the first features using a dimensionality reduction method;
creating, by the electronic device, a function associated with learned data corresponding to the selected at least one toxicity feature for determining the toxicity of the at least one biochemical compound data, wherein creating the function comprise computing a mathematical function derived by linear combination of the stored at least one second feature retrieved from the database; and
storing, by the electronic device, the created function to determine the toxicity of subsequent at least one biochemical compound data in the database associated with the electronic device.

4. The method of claim 3, wherein the method further comprises:
receiving, by the electronic device, the input corresponding to the at least one biochemical compound data;
inserting, by the electronic device, combination of the features corresponding to the at least one biochemical compound data, in the stored function; and
determining, by the electronic device, the toxicity of the at least one biochemical compound data, based on inserting combination of the appropriate features in the stored function.

5. The method of claim 1, wherein the method further comprises analyzing, by the electronic device, reaction data of the toxin degradation data, toxin synthesis data, and/or toxin route data, wherein the reaction data comprises reaction level differences in pathways between a strain associated with organisms and a strain associated with the different organism, corresponding to the compound.

6. The method of claim 5, wherein the strain comprises variation of a species associated with the organism and a species associated with the different organism.

7. The method of claim 3, wherein the mathematical function is derived using at least one of a Random Forest Method (RFM) and a Support Vector Machine (SVM) method.

8. An apparatus for assessing ability of an organisms to metabolize at least one toxic compound, in a computing environment comprising:
a processor; and
a memory unit coupled to the processor, wherein the memory unit comprises a processing module configured to:
receive an input corresponding to at least one biochemical compound data, wherein the at least one biochemical compound data comprises compound data, at least one reaction data, and at least one pathway data;
extract compound data associated with the received at least one reaction data and the at least one pathway data;
retrieve molecular information corresponding to the extracted compound data from a database associated with the electronic device;
generate a plurality of a first features corresponding to the retrieved molecular information, wherein generating the plurality of the first features comprises identifying at least one of constitutional data, topological data, electronic data, and fingerprint data;
identify toxic data of the at least one biochemical compound data based on mapping the generated first features with at least one of a set of second features stored in the database associated with the electronic device; and
assess an effect of toxicity of the compound data on the at least one reaction data and the at least one pathway data, based on the identified toxic data, wherein assessing the effect of toxicity comprise determining the lethality of the compound data to at least one of, an organism, a strain of the organism and a strain of a different organism.

9. The apparatus of claim 8, wherein the processing module is further configured to:
determine at least one of toxin degradation data, toxin synthesis data, and toxin route data corresponding to the at least one reaction data and the at least one pathway data, based on the identified toxic data;
analyze the toxin degradation data, toxin synthesis data, and/or toxin route data;
output ability data indicating the ability of the at least one biochemical compound data to degrade the at least one toxic compound, based on the analyzed toxin degradation data, toxin synthesis data, and/or toxin route data;
output, by the electronic device, the ability data indicating the ability of the at least one biochemical compound data to synthesize the at least one toxic compound, based on the analyzed toxin degradation data, toxin synthesis data, and/or toxin route data, wherein outputting the ability data comprise determining the capability of the at least one biochemical compound data to metabolize the at least one toxic compound, wherein outputting the ability data comprise determining an existence of the route between the compounds; and
output a suggestion data corresponding to an alternative reaction route within the provided at least one biochemical compound data, wherein the suggestion data is outputted based on the analyzed toxin degradation data, toxin synthesis data, and/or toxin route data corresponding to the at least one biochemical compound data, wherein outputting the suggestion data comprise identifying at least one non-toxic route between the compounds.

10. The apparatus of claim 8, wherein the processing module is further configured to:
select at least one toxicity feature from the generated plurality of the first features corresponding to the at least one biochemical compound data, wherein selecting the at least one toxicity feature comprises reducing the first features using a dimensionality reduction method;
create a function associated with learned data corresponding to the at least one toxicity feature for determining the toxicity of the at least one biochemical compound data, wherein creating the function comprises computing a mathematical function derived by linear combination of the stored at least one second feature retrieved from the database; and
store in the database the created function to determine the toxicity of subsequent the at least one biochemical compound data.

11. The apparatus as claimed in claim 8, wherein the processing module is further configured to:
receive the input corresponding to the at least one biochemical compound data;
insert combination of the appropriate features corresponding to the at least one biochemical compound data, in the stored function; and
determine the toxicity of the at least one biochemical compound data, based on inserting combination of the appropriate features in the stored function.

12. The apparatus of claim 8, wherein the processing module is further configured to analyze reaction data of the toxin degradation data, toxin synthesis data, and/or toxin route data, wherein the reaction data comprises a reaction level differences in pathways between a strain associated with organisms and a strain associated with the different organism, corresponding to the compound.

13. The apparatus of claim 12, wherein the strain comprises variation of a species associated with the organism and a species associated with the different organism.

14. The apparatus of claim 10, wherein the mathematical function is derived using at least one of a Random Forest Method (RFM) and a Support Vector Machine (SVM) method.
